(19) United States
(12) Patent Application Publication
(10) Pub. No.: US 2015/0066093 A1
(43) Pub. Date: Mar. 5, 2015

(54) PROXIMAL TIBIAL POSTERIOR PLATEAU LOCKING COMPRESSION PLATE

(71) Applicant: KUAN-YU LIN, KAOHSIUNG CITY (TW)

(72) Inventor: KUAN-YU LIN, KAOHSIUNG CITY (TW)

(21) Appl. No.: 14/011,950

(22) Filed: Aug. 28, 2013

(51) Int. Cl. A61B 17/80

(52) U.S. Cl. CPC A61B 17/8014; A61B 17/8057
USPC 606/291

(57) ABSTRACT

A proximal posterior tibial plateau locking compression plate is revealed. This locking compression plate mainly includes a fixing plate and a pressing head having at least one threaded fixing hole and bent forward from the fixing plate. At least one lateral threaded fixing hole is arranged along the two sides the fixing plate and a plurality of lower threaded fixing holes is disposed on the fixing plate, extending downward. The threaded fixing hole on the pressing head, each lateral threaded fixing hole and each lower threaded fixing hole are mounted with a threaded fastener. Thereby the fractured bone fragments on the rear side of the proximal tibia, including those avulsed by the posterior cruciate ligament, are reduced anatomically and fixated rigidly. This locking compression plate not only allows the surgical staff to treat the fractured bone conveniently but also enables the patient to regain their pre-injury activities smoothly.

### BACKGROUND OF THE INVENTION

1. Fields of the invention

The present invention relates to a proximal tibial posterior plateau locking compression plate that can be used to reduce the fractured posterior tibial plateau bone fragments, including those avulsed by the posterior cruciate ligament (PCL), back to their anatomical places and to fixate them rigidly.

2. Descriptions of Related Art

Human skeletal system is composed of connective tissues including bones, cartilages, tendons and ligaments. The skeletal system provides a rigid framework that supports our body as well as protects the internal organs and soft tissues. Moreover, all the components of skeletal system, together with the attached muscles, work synergistically to give to us the ability to perform various movements.

The knee joint is the largest joint in the human body and is formed as a pivotal-hinge joint by several bones including femur, tibia, fibula, and patella, which enable the knee to have flexion, extension, and slight medial and lateral rotation. The meniscii of the knee joint are two semicircular pads of fibrocartilage with a thickness of 3-9 mm between the tibia and the femur. The meniscii work as cushion pads to alleviate vertical pressure and make the joint surface slide easily over each other during the movement. Moreover, the knee is stabilized by several ligaments, which are tough bands of tissues that connect bones, including the anterior cruciate ligament (ACL) which protects the tibia from being pushed anteriorly, the posterior cruciate ligament (PCL) which prevents posterior displacement of the tibia, the lateral collateral ligament (LCL) protects the lateral side from an inside bending force, and the medial collateral ligament (MCL) prevents the knee from being bent open medially by a stress applied to the lateral side of the knee. Bones and ligaments work together to give the knee joint its ability to flexion, extension, as well as slight medial and lateral rotation. Since the knee joint supports nearly the entire weight of the body and is highly mobile, it is very vulnerable to acute traumatic injury as well as chronic wear and tear.

The knee is an intricate joint with numerous tendinous, ligamentous and meniscal attachments, which make it particularly vulnerable to complex injuries after trauma. Tibial plateau is the proximal weight-bearing part of tibia, or the shinbone, and can be divided into four parts: the medial plateau, the lateral plateau, the central eminence, and the posterior plateau. Therefore, tibial plateau fracture is a specific injury in which there is a fracture or break in the continuity at the proximal part of the tibia, and is often caused by a varus (inwardly angulating) or valgus (outwardly angulating) force combined with an axial loading or weight bearing on the knee. Injury patterns dictate whether the tibial plateau fracture is accompanied by other soft tissue injuries, such as meniscal or ligamentous tears.

The posterior cruciate ligament (PCL) is one of the four stabilizing ligaments of the knee joint as mentioned above. It extends from the top-rear surface of the tibia to bottom-front surface of the femur. Thus it functions as the posterior constraint of the knee joint restricting forces pushing the tibia posteriorly relative to the femur. The most common mechanism of avulsion fractures of the PCL at its tibial insertion site is a dashboard injury, in which a posteriorly directed force is applied to the pretibial area of a flexed knee causing the PCL along with a piece of a bone to be detached. PCL avulsion fracture can occur as an isolated injury (as shown in FIG. 5), or as often does, it can occur in concomitant with a complex tibial plateau fracture. If there is no proper fixing device used in time to reduce and fixate the PCL avulsed fragment back to its anatomical site, the injured knee joint would remain dynamically unstable, even if the concomitant tibial plateau fracture had fully healed, which would then progressively cause the knee to undergo early degeneration, namely the post-traumatic osteoarthritis of the knee joint. In prevention of such serious sequela, the patient often has to undergo a salvaging PCL reconstruction surgery utilizing either an allograft, which risks higher infection and non-healing rates, or an autologous graft, which is made of tendons taken from other parts of the patient's body, which in turn may have negative effects on his/her physical activities in the future.

To the best of our knowledge, an anatomical orthopedic plate that can be specifically applied to reduce and fixate the posterior part of the tibial plateau has yet to be designed. And this invention is the very first of its kind not only to optimally reduce and fixate a comminuted fracture of posterior tibial plateau, but also to treat the rather challenging PCL avulsion fracture with its special contour.

### SUMMARY OF THE INVENTION

Therefore it is the primary objective of the present invention to provide a proximal tibial posterior plateau locking compression plate that is specifically constructed to reduce and fixate a PCL avulsed bone fragment with a pressing head projecting and bent forward from a fixing plate which can be used to anatomically reduce and fixate the posterior tibial plateau fracture. It is believed that, with the advent of this newly developed bone plate, patients with such a difficult fracture pattern can be better treated and return to their daily activities earlier.

In order to achieve the above objective, a proximal posterior tibial plateau locking compression plate of the present invention mainly includes a fixing plate with a forward-bending pressing head projecting from the top of the fixing plate. The pressing head is composed of at least one threaded fixing hole. At least one lateral threaded fixing holes is arranged along two sides of the fixing plate and a plurality of lower threaded fixing holes extending downwards is disposed on the fixing plate. The threaded fixing hole of the pressing head, lateral threaded fixing holes and lower threaded fixing holes can be mounted with a threaded fastener.

The pressing head is connected to the fixing plate by a neck part.

The pressing head is directly extended and projecting from the fixing plate.

The lateral threaded fixing hole located most outside on each of two sides is inclined toward outside.

A plurality of auxiliary positioning holes is penetrating and mounted on the fixing plate.

### BRIEF DESCRIPTION OF THE DRAWINGS

The structure and the technical means adopted by the present invention to achieve the above and other objectives can be best understood by referring to the following detailed description of the preferred embodiments and the accompanying drawings, wherein

FIG. 1 is a perspective view of an embodiment according to the present invention;

FIG. 2 is a perspective view of another embodiment according to the present invention;

FIG. 3 is an embodiment in use according to the present invention;

FIG. 4 is a schematic drawing showing a cross sectional view of an embodiment in use according to the present invention;

FIG. 5 is a schematic drawing showing a broken bone on a top-rear surface of the tibia.

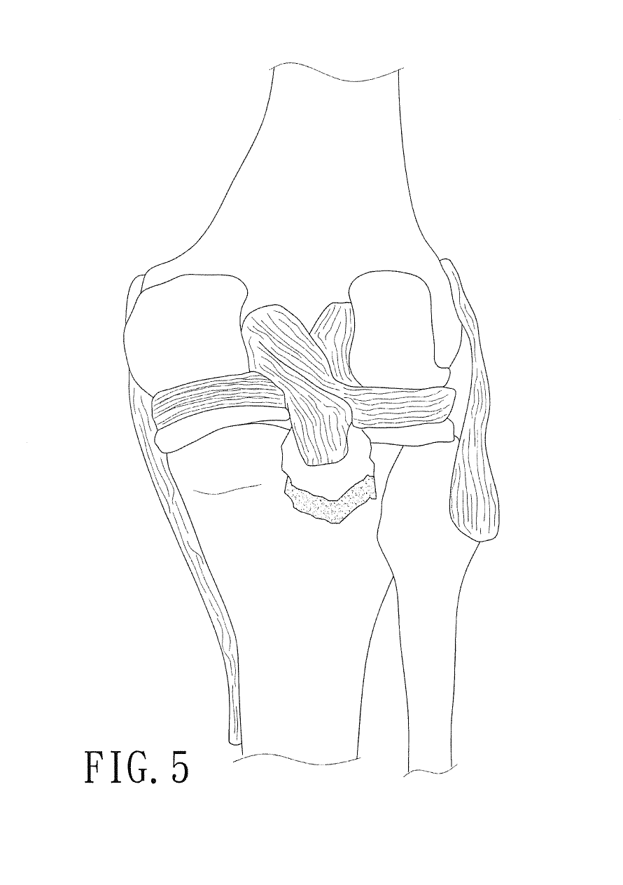

### DETAILED DESCRIPTION OF THE PREFERRED EMBODIMENT

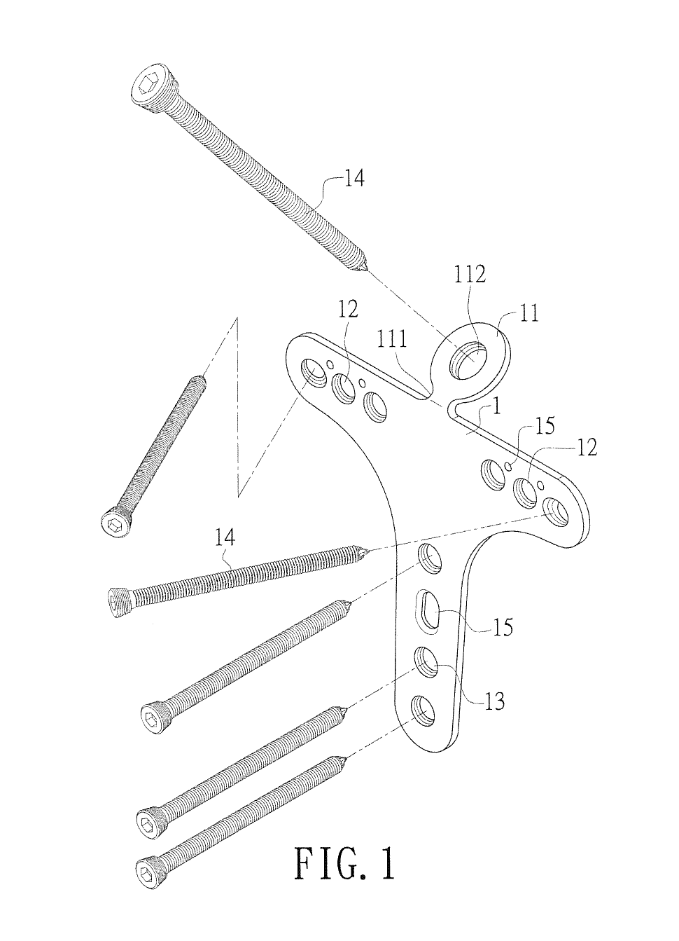

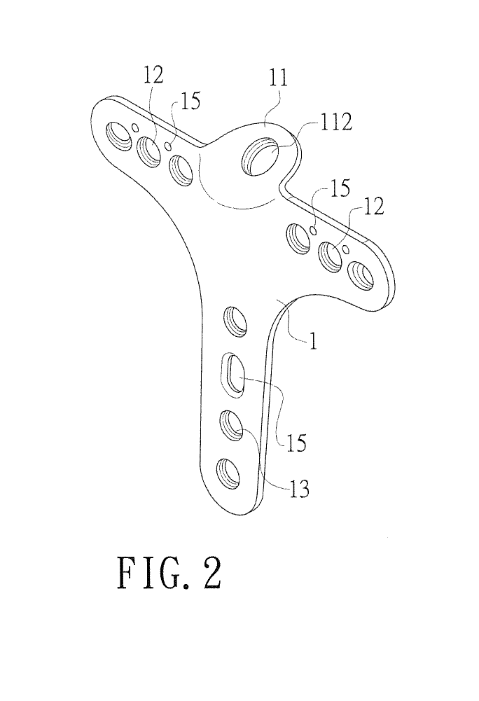

Refer to FIG. 1, the present invention is mainly a fixing plate 1, which can be made of metal, such as stainless steel or titanium. A pressing head 11 is projecting from the fixing plate 1. The pressing head 11 is connected to the fixing plate 1 by a neck part 111, as shown in FIG. 1 or is directly extended and projecting from the top end of the fixing plate 1, as shown in FIG. 2. The pressing head 11 is bent forward and is tilted at an angle of certain degrees. And at least one threaded fixing hole 112 is disposed on the pressing head 11 while at least one lateral threaded fixing hole 12 is arranged at each of two sides of the fixing plate 1, extending outward along each side of the fixing plate 1. The lateral threaded fixing hole 12 located most outside on each of two sides is inclined toward outside. Moreover, a plurality of lower threaded fixing holes 13 is disposed on the fixing plate 1, extending downward. The threaded fixing hole 112 on the pressing head, the lateral threaded fixing holes 12 and the lower threaded fixing holes 13 can be mounted with a threaded fastener 14 respectively. A plurality of auxiliary positioning holes 15 is penetrating and mounted on the fixing plate 1.

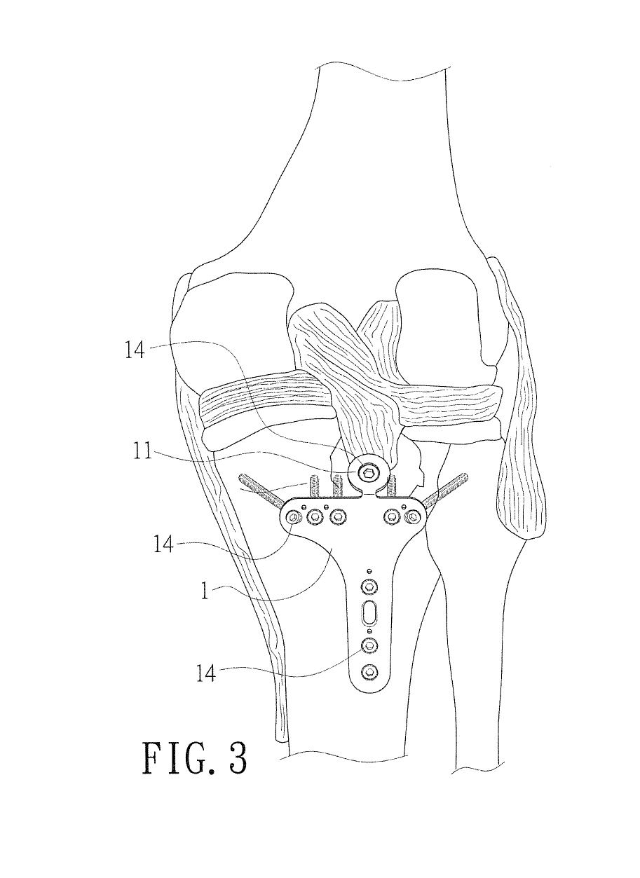

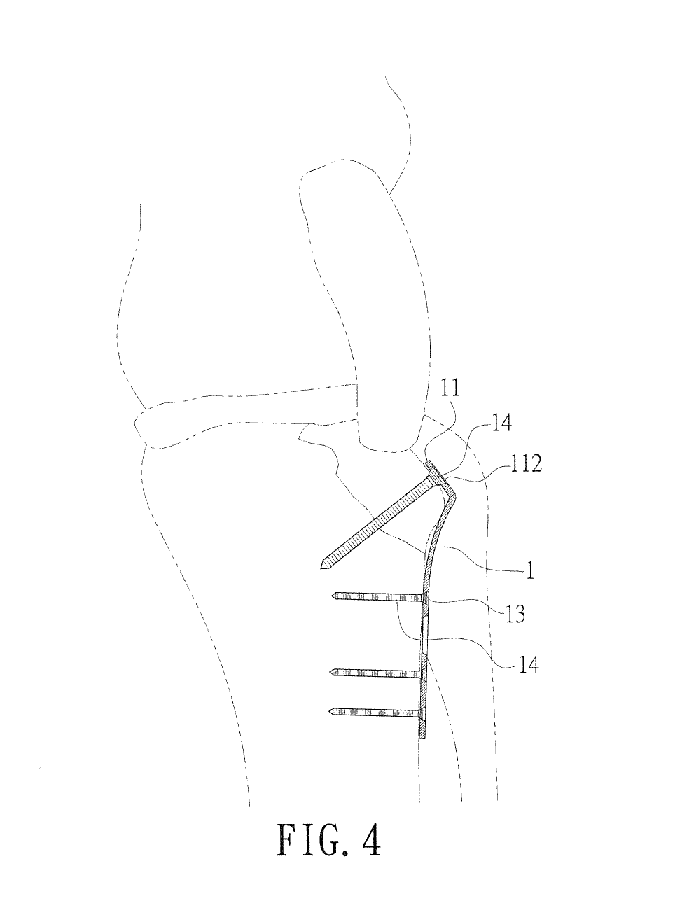

Refer to FIG. 3 and FIG. 4, once fracture or breakage occurs at the connection area between the posterior cruciate ligament (PCL) and the rear side of the proximal tibia, the fixing plate 1 is applied to the rear side of the proximal tibia anatomically to the contour of the bone. The pressing head 11 is used to press the bone fragments torn and displaced by the PCL back into place and hold the bone fragments in place. Now because the angle of the press head 11 has been pre-tilted, the press head 11 anatomically matches the shape of the rear side of the proximal tibia and holds the torn and displaced bone fragments in place precisely. First position the fixing plate 1 on the rear side of the proximal tibia by an auxiliary positioning component passing through each auxiliary positioning holes 15. Then the threaded fastener 14 is passing through each of the threaded fixing holes 112, the lateral threaded fixing holes 12, and the lower threaded fixing holes 13 and threaded into the tibia so as to arrange and position the fixing plate 1 stably on the rear end of the proximal tibia. By the press head 11 that puts the PCL avulsed bone fragments back into place and holds them together precisely and stably, the bone fragments and the fractured tibia are connected and restored. The present invention helps the tibia regain its shape anatomically and patients can return to their pre-injury activities.

In summary, with a design unmatched by any other currently available bone plate, the present invention can hold the bone fragments of the posterior tibial plateau in place precisely by a fixing plate, which is equipped with a pressing head to perfectly reduce the bone fragment specifically avulsed by the posterior cruciate ligament. With the utilization of this novel orthopedic plate, the surgical staff can easily obtain a satisfactory anatomic reduction and fixation of the posterior tibial plateau fracture, even in its most serious type; as a result, the injured limb can regain its normal function and patients can return to their pre-injury activities sooner. Hence with these aided benefits, we believe this novel plate can bring a higher practical value to both the medical professionals as well as the patient.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A proximal tibial plateau posterior locking compression plate comprising:
a fixing plate disposed with at least one lateral threaded fixing holes extending outward along each of two sides thereof and a plurality of lower threaded fixing holes extending downward; and
a pressing head projecting from top of the fixing plate, bent forward from the fixing plate and being arranged with at least one threaded fixing hole; wherein the at least one threaded fixing hole, the at least one lateral threaded fixing holes and each of the lower threaded fixing holes are engaged with a threaded fastener therein.

2. The device as claimed in claim 1, wherein the pressing head is connected to the fixing plate by a neck part.

3. The device as claimed in claim 1, wherein the pressing head is directly extended and projecting from a top end of the fixing plate.

4. The device as claimed in claim 1, wherein the lateral threaded fixing hole located most outside on each of two sides is inclined toward outside.

5. The device as claimed in claim 1, wherein a plurality auxiliary positioning holes is penetrating and mounted on the fixing plate.
